(19) United States
(12) Patent Application Publication
(10) Pub. No.: US 2011/0137033 A1
(43) Pub. Date: Jun. 9, 2011

(54) PROCESS FOR PREPARING (4,6-DIMETHYLPYRIMIDIN-2-YL)PHENYLAMINE (PYRIMETHANIL)

(75) Inventors: HARALD WINSEL, Freinsheim (DE); Wolfgang Ladner, Fussgonheim (DE); Ralph Busch, Worms (DE)

(73) Assignee: BASF SE, Ludwigshafen (DE)

(21) Appl. No.: 12/959,476

(22) Filed: Dec. 3, 2010

(30) Foreign Application Priority Data
Dec. 4, 2009 (EP) 09178094.0

(51) Int. Cl. C07D 239/28

(52) U.S. Cl. 544/297

(57) ABSTRACT

A process for preparing (4,6-dimethylpyrimidin-2-yl)phenylamine (pyrimethanil) of the formula

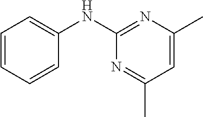

by reacting aniline with cyanamide in the presence of an aqueous acid to give the corresponding phenylguanidinium salt and reacting the phenylguanidinium salt with acetylacetone in the presence of an aqueous base, wherein the process is performed as a one-pot process, by not isolating the phenylguanidinium salt formed as an intermediate.

The invention relates to a process for preparing (4,6-dimethylpyrimidin-2-yl) phenylamine (pyrimethanil) by reacting aniline with cyanamide in the presence of an aqueous acid to give the corresponding phenylguanidinium salt and reacting the phenylguanidinium salt with acetylacetone in the presence of an aqueous base.

Pyrimethanil (see the formula below) is of significance as a fungicide and is utilized in various formulations and active ingredient combinations.

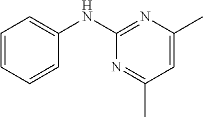

According to A. Kreutzberger et al., Arch. Pharm (Weinheim) 318, 1043-45 (1985), particular 2-(methylthioanilino)pyrimidines are obtained by reacting corresponding guanidinium nitrates with 2,4-pentanedione in aqueous ethanol.

EP 717 038 A1 (Hoechst Schering AgrEvo GmbH) relates to a process for preparing particular 2-anilino-pyrimidine derivatives by reacting phenylguanidinium carbonates with 1,3-diketones under reduced pressure while removing the water and carbon dioxide formed.

According to WO 03/070708 A1 (Degussa AG) and literature cited therein, pyrimethanil is synthesized by the following process:

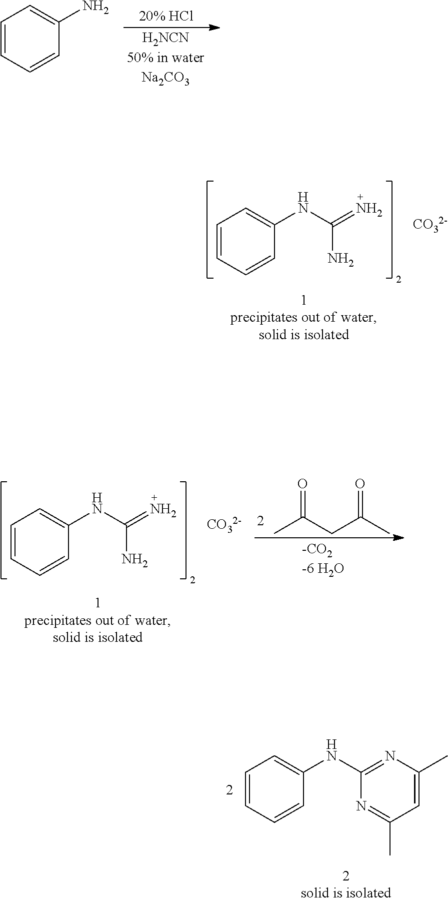

The guanidinium carbonate intermediate 1 is isolated, which achieves a significant purifying effect. The isolated solid is reacted in a second reaction with acetylacetone to give pyrimethanil 2, which is isolated after crystallization.

The syntheses described have at least two stages, requiring performance of two solid isolation steps.

It was an object of the present invention to overcome the disadvantages of the prior art and provide an improved, economically viable process for preparing (4,6-dimethylpyrimidin-2-yl)phenylamine (pyrimethanil). The preparation process should additionally be particularly simple and efficient to perform and lead to the product in high purity and yield.

Accordingly, a process has been found for preparing (4,6-dimethylpyrimidin-2-yl) phenylamine (pyrimethanil) by reacting aniline with cyanamide in the presence of an aqueous acid to give the corresponding phenylguanidinium salt and reacting the phenylguanidinium salt with acetylacetone in the presence of an aqueous base, which comprises performing the process as a one-pot process, by not isolating the phenylguanidinium salt formed as an intermediate.

The synthesis of pyrimethanil has been improved by obtaining the product in a one-stage reaction regime (one-pot process) with only one solid isolation step. Only at the stage of the end product is a solid isolation necessary.

It has been recognized in accordance with the invention that neither the intermediate of the corresponding phenylguanidinium salt nor a phenylguanidinium salt obtainable by salt exchange need be isolated. It has been found that this intermediate step, considered to be necessary in the art, can be dispensed with.

It has been found that, surprisingly, the one-pot reaction regime with only one solid isolation step gives at least the same yield and especially quality as a two-stage reaction regime with intermediate isolation of a phenylguanidinium salt. The reduced number of solid isolation steps in accordance with the invention does not reduce the purity of the pyrimethanil obtained.

The yield is ≧80% particularly ≧81 to 82%, and the purity after workup by crystallization from a solvent is ≧97.5%, particularly ≧97.6 to 97.8% (HPLC with internal standard), or ≧99.8%, particularly ≧99.9 to 99.93% (GC area percent) (for GC and HPLC methods see the example below).

The process according to the invention leads, after reaction of aniline with cyanamide in the acidic pH range, to the phenylguanidinium salt solution, which then leads directly with acetylacetone (=2,4-pentanedione) in the one-pot process in the alkaline pH range to (4,6-dimethylpyrimidin-2-yl)phenylamine (pyrimethanil).

The reaction of aniline with cyanamide is preferably performed at a pH in the range from 1.2 to 5.0, particularly 2.0 to 3.5.

The reaction of the phenylguanidinium salt with acetylacetone is preferably performed at a pH in the range from 5.0 to 12.0, particularly 7.8 to 8.1.

The amount of acetylacetone excess may be such that after the conversion to the target product, any by-products formed can surprisingly be removed as the lower phase with the amounts of water introduced into or formed in the synthesis. A time-consuming azeotropic removal of the amounts of water therefore becomes superfluous.

Thereafter, any acetylacetone excess present can be distilled off and used in subsequent batches.

The reactants are preferably used in a molar ratio range of aniline:cyanamide:acetylacetone=1.0:0.8-2.0:0.8-10.0, particularly 1.0:1.0-1.2:1.5-2.5.

The acid used in the process according to the invention is preferably HCl, H₂SO₄, HNO₃, H₃PO₄, HCOOH, CH₃COOH or a C₃₋₈-carboxyilic acid. Examples of a C₃₋₈-carboxylic acid are propionic acid, n-butanoic acid, isobutanoic acid, n-pentanoic acid, n-hexanoic acid, n-octanoic acid, ethylhexanoic acid.

Particularly preferred acids are HCl (hydrochloric acid) and H₂SO₄ (sulfuric acid). In particular, the acid used is not H₂CO₃ (carbonic acid).

The base used in the process according to the invention is preferably NaOH, KOH, Ca(OH)₂ or a tertiary amine base. Examples of a tertiary amine base are trimethylamine, triethylamine, tri-n-propylamine, diisopropylethylamine, tri-n-butylamine, N-methylmorpholine, pyridine.

Particularly preferred bases are NaOH (sodium hydroxide solution) and KOH (potassium hydroxide solution).

The one-pot process according to the invention is preferably performed in the presence of 100 to 800% by weight, particularly 150 to 250% by weight, of water, based in each case on aniline used. The water can be added correspondingly and/or may result from the feedstocks.

The process is preferably performed within a temperature range from 30 to 100° C., particularly 70 to 90° C.

The process is preferably performed within an absolute pressure range from 0.5 to 2 bar, particularly 0.9 to 1.1 bar.

In particular in the process according to the invention, completion of reactions is followed by removal of the aqueous phase and distillative removal of unconverted acetylacetone. The distillation bottoms then comprise the product of value.

More particularly pure pyrimethanil is finally obtained from the crude product by crystallization from isopropanol, n-propanol, methanol, ethanol, n-butanol, 2-butanol, isobutanol, C₅₋₁₀ alcohol or acetonitrile.

Examples of a C₅₋₁₀ alcohol are n-pentanol, n-hexanol, 2-ethylhexanol, n-octanol.

The solvents mentioned may also be mixtures with water. Particularly preferred solvents are isopropanol and ethanol.

### EXAMPLE

### Preparation of pyrimethanil=(4,6-dimethylpyrimidin-2-yl)phenylamine

| | | | |
|---|---|---|---|
| Batch: | 186.6 g | 2.00 mol | aniline |
| | 201.8 g | 2.40 mol | cyanamide solution, 50% |
| | | | by weight in water |
| | 213.5 g | 1.87 mol | aqueous hydrochloric acid, |
| | | | 32% by weight |
| | 497.0 g | 4.96 mol | acetylacetone |
| | 206.8 g | 2.59 mol | aqueous sodium hydroxide |
| | | | solution, 50% by weight |
| | 500 g | | isopropanol |

A nitrogen-inertized flange reactor was initially charged with aniline and 50% cyanamide solution in water. Subsequently, 32% hydrochloric acid was metered in up to a pH of 2.5. After the metered addition of HCl, the mixture was heated from internal temperature 45° C. to 95° C. within two hours. During the heating phase the pH was kept at 2.5 by adding further hydrochloric acid. Subsequently, acetylacetone was pumped in at internal temperature 80° C., and 50% sodium hydroxide solution was metered in up to a pH of 7.9. After a continued stirring period of one hour, the lower aqueous phase was removed. The acetylacetone excess of the upper phase was distilled off at a pressure of 750-35 mbar up to a maximum bottom temperature of 125° C. The acetylacetone distilled off can be used in subsequent batches.

The bottoms were cooled to 80° C. and admixed with isopropanol. This was followed by cooling gradually to internal temperature of 0° C., and filtration of the product slurry with suction through a glass filter crucible. The reactor was rinsed through with water, which was filtered with suction. The product was washed twice with isopropanol and dried in a vacuum drying cabinet at 70° C. and 10 mbar.

The isolated yield was 324 g=1.626 mol=81.3% of theory at a product purity of 97.6% (HPLC with internal standard*)) or 99.9% (GC area %**)). **) GC: 30 m DB 1701* 0.32 mm, film thickness 0.25 μm, injector 250° C., detector (FID) 300° C., injection volume 0.2 μl (0.1 g/liter of methanol), program: 50° C. (2 min), 10° C./min to 250° C. (8 min).*) HPLC: 1100 series from Agilent; Nucleosil column 120-5 C18 250×4 mm; instrument parameters: injection volume 5 μl, flow rate 1 ml/min, temperature RT, column pressure 90 bar, detector wavelength 268 nm, chromatography time 8 min; mobile phase: 250 ml of Millipore water, 750 ml of acetonitrile, 1 g of ammonium acetate; sample preparation phase: 250 ml of Millipore water, 750 ml of acetonitrile; pyrimethanil standard with known content.

1.-11. (canceled)

12. A process for preparing (4,6-dimethylpyrimidin-2-yl)phenylamine (pyrimethanil) by reacting aniline with cyanamide in the presence of an aqueous acid to give the corresponding phenylguanidinium salt and reacting the phenylguanidinium salt with acetylacetone in the presence of an aqueous base, which comprises performing the process as a one-pot process, by not isolating the phenylguanidinium salt formed as an intermediate.

13. The process according to claim 12, wherein the reactants are used in a molar ratio range of aniline:cyanamide:acetylacetone=1.0:0.8-2.0:0.8-10.0.

14. The process according to claim 12, wherein the acid used is HCl, H₂SO₄, HNO₃, H₃PO₄, HCOOH, CH₃COOH or a C₃₋₈-carboxylic acid.

15. The process according to claim 12, wherein the base used is NaOH, KOH, Ca(OH)₂ or a tertiary amine base.

16. The process according to claim 12, wherein the one-pot process is performed in the presence of 100 to 800% by weight of water, based on aniline used.

17. The process according to claim 12, wherein the reaction of aniline with cyanamide is performed at a pH in the range from 1.2 to 5.0.

18. The process according to claim 12, wherein the reaction of the phenylguanidinium salt with acetylacetone is performed at a pH in the range from 5.0 to 12.0.

19. The process according to claim 12, wherein the one-pot process is performed within a temperature range from 30 to 100° C.

20. The process according to claim 12, wherein the one-pot process is performed within an absolute pressure range from 0.5 to 2 bar.

21. The process according to claim 12, wherein the aqueous phase is subsequently removed and unconverted acetylacetone is distilled off.

22. The process according to claim 21, wherein pure pyrimethanil is subsequently obtained by crystallization from in each case aqueous or non-aqueous isopropanol, n-propanol, methanol, ethanol, n-butanol, 2-butanol, isobutanol, C₅₋₁₀alcohol or acetonitrile.
